(19) United States
(12) Patent Application Publication
(10) Pub. No.: US 2026/0344972 A1
(43) Pub. Date: Nov. 5, 2026

(54) CONTROL APPARATUS, ENDOSCOPE SYSTEM, AND METHOD OF MEASURING PRESSURE INSIDE BODY

(71) Applicants: Haruhiro INOUE, Tokyo (JP); OLYMPUS MEDICAL SYSTEMS CORP., Tokyo (JP)

(72) Inventors: Haruhiro INOUE, Tokyo (JP); Yuma KASUYA, Tokyo (JP); Takefumi UESUGI, Tokyo (JP)

(73) Assignees: Haruhiro INOUE, Tokyo (JP); OLYMPUS MEDICAL SYSTEMS CORP., Tokyo (JP)

(21) Appl. No.: 19/712,296

(22) Filed: Jun. 18, 2026

Related U.S. Application Data
(63) Continuation of application No. PCT/JP2023/045548, filed on Dec. 19, 2023.

(51) Int. Cl. A61B 1/00; A61B 1/018

(52) U.S. Cl. CPC A61B 1/00004; A61B 1/00045; A61B 1/018

(57) ABSTRACT

A control apparatus includes: a processor comprising hardware, the processor being configured to acquire second pressure in a tube of an endoscope when first pressure in the tube is changed to remove a foreign object from inside the tube, acquire an elapsed time in which the second pressure in the tube meets a predetermined condition, and determine whether or not the foreign object in the tube is removed based on the second pressure in the tube and the elapsed time.

### CROSS REFERENCES TO RELATED APPLICATIONS

This application is a continuation of International Application No. PCT/JP2023/045548, filed on Dec. 19, 2023, the entire contents of which are incorporated herein by reference.

### BACKGROUND

### 1. Technical Field

The present disclosure is related to a control apparatus, an endoscope system, and a method of measuring pressure inside a body.

### 2. Related Art

In the related art, an endoscope system that is used for a diagnosis (hereinafter, described as a GERD diagnosis) for gastroesophageal reflux disease that is a disease in which a function of a cardiac part of the stomach decreases and gastric juices reflux into the esophagus is known (for example, see International Publication No. 2021/166127).

In the endoscope system described in International Publication No. 2021/166127, gas is supplied into the lumen via a pressure measurement tube that is arranged in an endoscope. Further, in the endoscope system, the GERD diagnosis is performed based on pressure change in the lumen that is measured while the gas is being supplied, and a relaxed state of the lower esophageal sphincter (LES) that is observed from an endoscopic image.

### SUMMARY

In some embodiments, a control apparatus includes: a processor comprising hardware, the processor being configured to acquire second pressure in a tube of an endoscope when first pressure in the tube is changed to remove a foreign object from inside the tube, acquire an elapsed time in which the second pressure in the tube meets a predetermined condition, and determine whether or not the foreign object in the tube is removed based on the second pressure in the tube and the elapsed time.

In some embodiments, an endoscope system includes: an endoscope that includes a first tube for measuring pressure inside a body; a second tube that is connected to the first tube; a pressure sensor that is connected to the second tube; and a control apparatus that includes a processor comprising hardware, the processor being configured to acquire second pressure in the first tube when first pressure in the first tube is changed to remove a foreign object from inside the first tube, acquire an elapsed time in which the second pressure in the first tube meets a predetermined condition, and determine whether or not the foreign object in the first tube is removed based on the second pressure in the first tube and the elapsed time.

In some embodiments, provided is a method of measuring pressure inside a body. The method includes: acquiring second pressure in a tube of an endoscope when first pressure in the tube is changed to remove a foreign object from inside the tube; acquiring an elapsed time in which the second pressure in the tube meets a predetermined condition; and determining whether or not the foreign object in the tube is removed based on the second pressure in the tube and the elapsed time.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to a first embodiment;

FIG. 2 is a flowchart illustrating a method of measuring pressure inside a body;

FIG. 3 is a diagram illustrating a pressure propagation state when a pressure generator is operated;

FIGS. 4A and 4B are diagrams for explaining a process of determining removal of residual liquid (Step S3);

FIGS. 5A and 5B are diagrams for explaining the process of determining removal of the residual liquid (Step S3);

FIGS. 6A and 6B are diagrams for explaining the process of determining removal of the residual liquid (Step S3);

FIG. 7 is a diagram illustrating an image that is displayed by a display unit at Step S4;

FIG. 8 is a diagram illustrating an image that is displayed by the display unit in a GERD diagnosis;

FIG. 9 is a diagram illustrating an endoscope system according to a second embodiment;

FIG. 10 is a diagram illustrating a pressure propagation state when a suction device performs suction operation;

FIGS. 11A and 11B are diagrams for explaining a process of determining removal of residual liquid (Step S3);

FIGS. 12A and 12B are diagrams for explaining the process of determining removal of the residual liquid (Step S3);

FIGS. 13A and 13B are diagrams for explaining the process of determining removal of the residual liquid (Step S3);

FIG. 14 is a diagram for explaining a process of determining removal of residual liquid (Step S3) according to a third embodiment;

FIGS. 15A and 15B are diagrams for explaining the process of determining removal of the residual liquid (Step S3) according to the third embodiment;

FIG. 16 is a diagram for explaining the process of determining removal of the residual liquid (Step S3) according to the third embodiment;

FIGS. 17A and 17B are diagrams for explaining the process of determining removal of the residual liquid (Step S3) according to the third embodiment;

FIG. 18 is a diagram illustrating an image that is displayed by a display unit at Step S4 according to a fourth embodiment;

FIG. 19 is a flowchart illustrating a method of measuring pressure inside a body according to a fifth embodiment;

FIG. 20 is a diagram illustrating an endoscope system according to a sixth embodiment;

FIG. 21 is a diagram illustrating a pressure propagation state when a gas supply device is operated;

FIG. 22 is a diagram illustrating an endoscope system according to a seventh embodiment; and

FIGS. 23A and 23B is a diagram for explaining a process of determining removal of residual liquid (Step S3).

### DETAILED DESCRIPTION

Modes (hereinafter, embodiments) for carrying out the present invention will be described below with reference to the drawings. The present invention is not limited by the embodiments described below. Further, in description of the drawings, the same components are denoted by the same reference symbols.

### First Embodiment

### Overall Configuration of Endoscope System

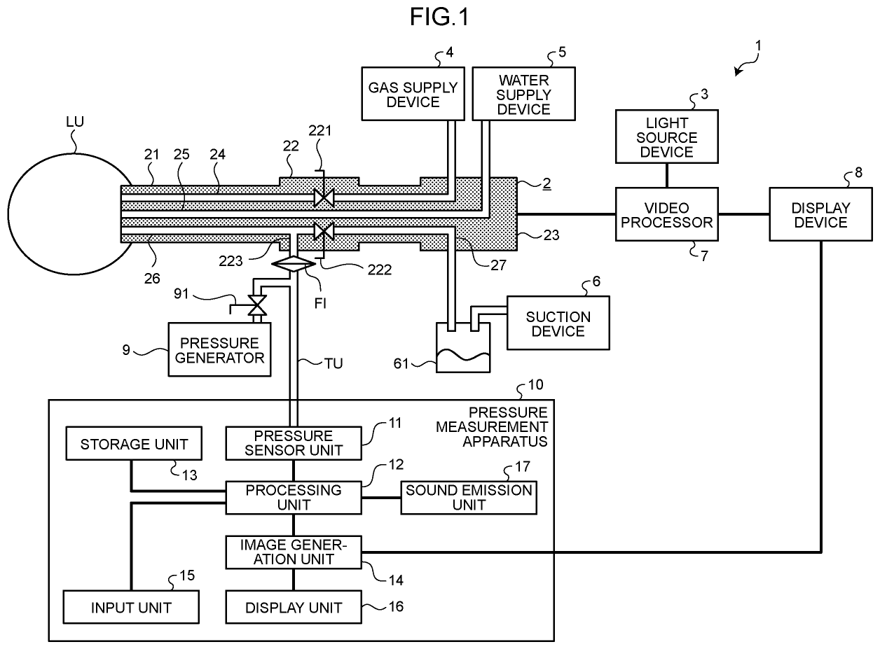

FIG. 1 is a diagram illustrating an endoscope system 1 according to a first embodiment.

The endoscope system 1 is a system for performing a diagnosis (hereinafter, described as a GERD diagnosis) for gastroesophageal reflux disease that is a disease in which a function of a cardiac part of the stomach decreases and gastric juices reflux into the esophagus. The endoscope system 1 includes, as illustrated in FIG. 1, an endoscope 2, a light source device 3, a gas supply device 4, a water supply device 5, a suction device 6, a video processor 7, a display device 8, a pressure generator 9, and a pressure measurement apparatus 10.

The endoscope 2 includes a portion that is inserted into a living body, performs imaging inside the living body, and outputs an image signal generated by the imaging. The endoscope 2 includes, as illustrated in FIG. 1, an insertion portion 21, an operation unit 22, and a connector 23.

Meanwhile, a “distal end side” described below indicates a distal end side of the insertion portion 21 (a distal end side in an insertion direction into a living body). Further, a “proximal end side” described below indicates a side separated from the distal end of the insertion portion 21.

The insertion portion 21 is a portion, at least a part of which is flexible, and which is inserted into the living body. At the distal end of the insertion portion 21, although specific illustration is omitted in the drawings, an illumination lens and an imaging unit are provided.

The illumination lens is a lens that applies illumination light, which is supplied from the light source device 3 and emitted from an emission end of a light guide (not illustrated) that is pulled around inside the endoscope 2, to the inside of a lumen LU (FIG. 1).

The imaging unit includes an objective optical system (not illustrated) that condenses light (object image) that is applied to the inside of the lumen LU and that is reflected inside the lumen LU, and an image sensor (not illustrated) that captures an object image that is formed by the objective optical system. Further, an image signal that is captured by the image sensor is transmitted to the video processor 7 via a signal cable (not illustrated) that is pulled around inside the endoscope 2.

The operation unit 22 is a part that is connected to the proximal end side of the insertion portion 21 and receives various kinds of operation from a doctor or the like. The operation unit 22 includes, as illustrated in FIG. 1, a gas supply button 221, a suction button 222, and a treatment tool insertion port 223.

The gas supply button 221 is a button that causes the gas supply device 4 to perform gas supply operation to the inside of the lumen LU when being turned on by a user, such as a doctor, and causes the gas supply device 4 to stop the gas supply operation when being turned off.

The suction button 222 is a button that causes the suction device 6 to perform fluid suction operation inside the lumen LU by being turned on by the user, such as the doctor, and causes the suction device 6 to stop the suction operation when being turned off.

The treatment tool insertion port 223 is an insertion port that communicates with a treatment tool tube 26 (FIG. 1) (to be described later) that is arranged inside the insertion portion 21, and used for inserting a treatment tool (not illustrated) into the treatment tool tube 26.

The connector 23 is a connector that is arranged on a proximal end side of the operation unit 22, and connected to the light source device 3 and the video processor 7.

The endoscope 2 described above includes, as illustrated in FIG. 1, a gas supply tube 24, a water supply tube 25, the treatment tool tube 26, and a suction tube 27.

The gas supply tube 24 is a tube that is extended from the connector 23 to the distal end of the insertion portion 21 via the operation unit 22, circulates gas that is supplied from the gas supply device 4, and discharges the gas from the distal end of the insertion portion 21.

The water supply tube 25 is a tube that is extended from the connector 23 to the distal end of the insertion portion 21 via the operation unit 22, circulates liquid that is supplied from the water supply device 5, and discharges the liquid from the distal end of the insertion portion 21.

The treatment tool tube 26 is a tube that is extended from the connector 23 to the distal end of the insertion portion 21 via the operation unit 22, and allows a treatment tool (not illustrated) that is inserted via the treatment tool insertion port 223 to be output from the distal end of the insertion portion 21.

The suction tube 27 is a tube that is extended from the connector 23 to the treatment tool tube 26 via the operation unit 22, and communicates with the treatment tool tube 26 when the suction button 222 is turned on. Further, the suction tube 27 circulates the liquid that is sucked by the suction device 6 from the distal end of the insertion portion 21 via the treatment tool tube 26, and guides the liquid to the suction device 6.

The light source device 3 supplies illumination light for illuminating the inside of the living body to an incident end of the light guide (not illustrated) that is pulled around inside the endoscope 2, via the connector 23. With this configuration, the illumination light is applied to the inside of the lumen LU from the illumination lens as described above via the light guide.

The gas supply device 4 adjusts pressure of gas that is supplied from a gas supply source (not illustrated, for example, a carbon dioxide cylinder) to predetermined pressure, and discharges the gas to the inside of the lumen LU from the distal end of the insertion portion 21 through the gas supply tube 24.

The water supply device 5 drives in accordance with operation of a foot switch (not illustrated), for example. Further, the water supply device 5 adjusts a flow rate of cleaning water that is stored in a tank (not illustrated) to a predetermined flow rate by using a built-in pump (not illustrated), and discharges the cleaning water to the inside of the lumen LU from the distal end of the insertion portion 21 through the water supply tube 25.

The suction device 6 generates negative pressure by a built-in vacuum pump (not illustrated), and sucks fluid in the lumen LU from the distal end of the insertion portion 21 through the suction tube 27 and the treatment tool tube 26. Further, the sucked fluid is discharged to a bottle unit 61 (FIG. 1).

The video processor 7 inputs an image signal from the imaging unit (image sensor) that is arranged on the distal end of the insertion portion 21. Further, the video processor 7 performs a predetermined process on the image signal to generate an endoscopic image, and causes the display device 8 to display the endoscopic image.

The display device 8 is a display using liquid crystal or the organic Electro Luminescence (EL), and displays the endoscopic image or the like that is generated by the video processor 7.

The pressure generator 9 is connected to a measurement tube TU that communicates with the treatment tool tube 26 via the treatment tool insertion port 223. In the first embodiment, the pressure generator 9 includes a syringe pump that pumps out a predetermined amount of gas in accordance with user operation. Meanwhile, the pressure generator 9 need not always include the syringe pump, but may include a syringe that sends out a predetermined amount of gas by manual operation. Further, the pressure generator 9 sends out a predetermined amount of gas to the inside of the lumen LU through the measurement tube TU and the treatment tool tube 26, and generates predetermined pressure. The measurement tube TU and the treatment tool tube 26 correspond to a pressure measurement tube.

Here, in the measurement tube TU, as illustrated in FIG. 1, a hydrophobic filter FI is arranged to prevent liquid from flowing into the measurement tube TU. Further, a manual valve 91 is arranged between the pressure generator 9 and the measurement tube TU as illustrated in FIG. 1. In a state in which the pressure generator 9 is connected, operation on the endoscope 2 may be disturbed. Therefore, when the pressure generator 9 is not to be used, it is preferable to close the manual valve 91 and remove the pressure generator 9. In other words, the manual valve 91 is arranged to ensure an airtight state of the measurement tube TU even when the pressure generator 9 is removed.

The pressure measurement apparatus 10 corresponds to a control apparatus. The pressure measurement apparatus 10 includes, as illustrated in FIG. 1, a pressure sensor unit 11, a processing unit 12, a storage unit 13, an image generation unit 14, an input unit 15, a display unit 16, and a sound emission unit 17.

The pressure sensor unit 11 is connected to the measurement tube TU, measures, by a pressure-sensitive element (not illustrated), pressure that propagates through the measurement tube TU, and outputs an electrical signal corresponding to the pressure to the processing unit 12, under the control of the processing unit 12. The pressure sensor unit 11 intermittently measures pressure with a period of about 1 to 100 milliseconds (ms).

The processing unit 12 corresponds to a processor. The processing unit 12 is implemented by causing a controller, such as a Central Processing Unit (CPU) or a Micro Processing Unit (MPU), to execute various programs that are stored in the storage unit 13, and comprehensively controls operation of the entire pressure measurement apparatus 10. Meanwhile, the processing unit 12 is not limited to the CPU or the MPU, but may include an integrated circuit, such as an Application Specific integrated Circuit (ASIC) or a Field-Programmable Gate Array (FPGA).

Functions of the processing unit 12 as described above will be described in the section of “method of measuring pressure inside body” to be described later.

The storage unit 13 stores therein a program that is executed by the processing unit 12, information that is needed for a process performed by the processing unit 12, or the like.

The image generation unit 14 generates a predetermined image under the control of the processing unit 12. Further, the predetermined image is displayed by the display unit 16 or the display device 8.

The input unit 15 is configured with an operation device, such as an input button and a touch panel, and receives user operation. Further, the input unit 15 outputs an operation signal corresponding to the user operation to the processing unit 12.

The display unit 16 is a display using liquid crystal or organic Electro Luminescence (EL), and displays an image or the like that is generated by the image generation unit 14.

The sound emission unit 17 is configured with a speaker or the like that outputs voice, and outputs predetermined voice under the control of the processing unit 12.

The display unit 16 and the sound emission unit 17 as described above correspond to a notification unit.

### Method of Measuring Pressure Inside Body

A method of measuring pressure inside a body by using the endoscope 2, the pressure generator 9, and the pressure measurement apparatus 10 as described above will be described below.

In the following, a process that is performed by the processing unit 12 will be described as the method of measuring pressure inside a body.

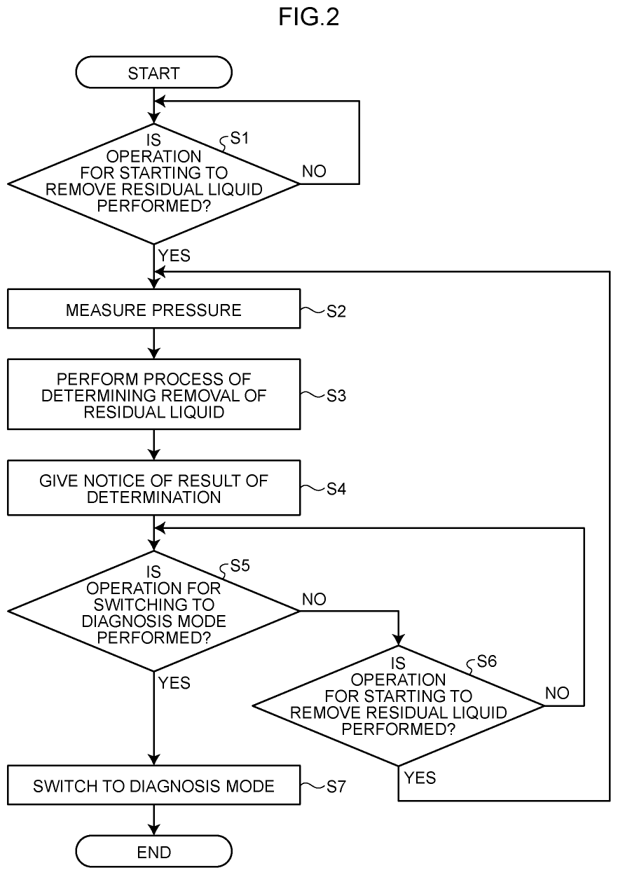

FIG. 2 is a flowchart illustrating the method of measuring pressure inside a body.

A doctor inserts the insertion portion 21 from a mouth of a patient to a lower esophageal sphincter (LES) that is located at a junction of the stomach and the esophagus. Further, a user, such as the doctor, performs operation on the input unit 15 for starting to remove residual liquid. Furthermore, the user, such as the doctor, opens the manual valve 91, performs user operation on the pressure generator 9 for causing the pressure generator 9 to operate, and, after a lapse of a certain time, performs user operation of stopping the operation of the pressure generator 9.

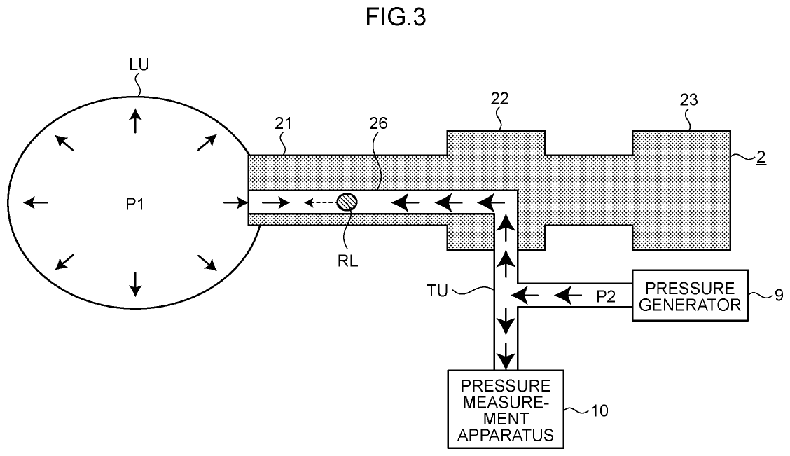

FIG. 3 is a diagram illustrating a pressure propagation state when the pressure generator 9 is operated. Specifically, solid arrows indicate propagation directions of pressure. Further, sizes of arrow feathers indicate pressure intensities. Furthermore, a dashed arrow indicates a movement direction of residual liquid RL. The residual liquid RL corresponds to a foreign object.

Here, immediately after the insertion portion 21 is introduced into the lumen LU, weak pressure P1 (normally, about 0 to 5 mmHg) is generated in the lumen LU. Further, when the pressure generator 9 generates pressure P2 (for example, equal to or larger than 20 mmHg) that is stronger than the pressure P1, the pressure propagates through the inside of the treatment tool tube 26 and the inside of the measurement tube TU, and is transmitted to the pressure measurement apparatus 10 and the residual liquid RL in the treatment tool tube 26. Accordingly, kinetic energy is generated on the residual liquid RL, so that the residual liquid RL moves to a lower-pressure side (the pressure P1 side).

The pressure measurement apparatus 10 is set to a “residual liquid removal monitoring mode” at the time of activation. The “residual liquid removal monitoring mode” is a mode in which whether or not operation of removing the residual liquid RL in the treatment tool tube 26 is appropriately performed is monitored.

Further, in the “residual liquid removal monitoring mode”, the processing unit 12 continuously monitors whether or not the user, such as the doctor, performs operation on the input unit 15 for starting to remove the residual liquid (Step S1).

When it is determined that the operation for starting to remove the residual liquid is performed (Step S1: Yes), the processing unit 12 causes the pressure sensor unit 11 to start to measure pressure (Step S2). Further, the processing unit 12 performs a process of determining removal of the residual liquid RL based on the pressure that is measured by the pressure sensor unit 11 (Step S3).

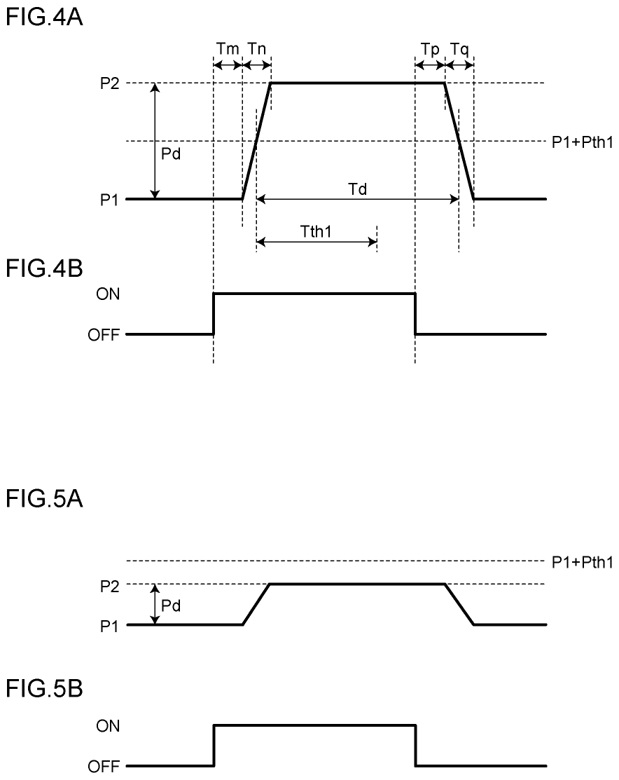

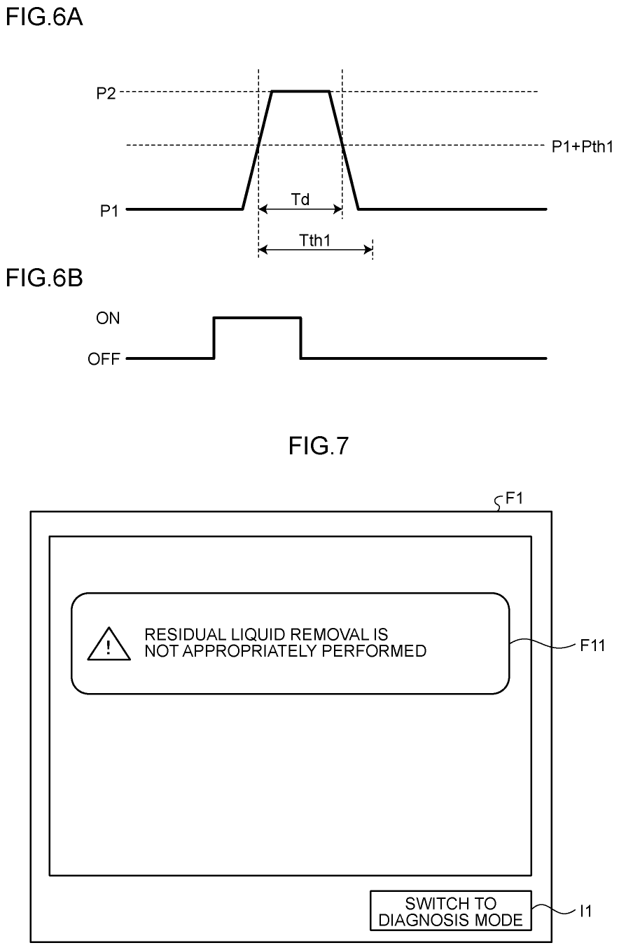

FIG. 4A to FIG. 6B are diagrams for explaining the process of determining removal of the residual liquid RL (Step S3). Specifically, FIG. 4A), FIG. 5A, and FIG. 6A are diagrams illustrating behaviors of the pressure that is measured by the pressure sensor unit 11 when the pressure generator 9 is operated. FIG. 4B, FIG. 5B, and FIG. 6B are diagrams illustrating operation states of the pressure generator 9, where “ON” indicates a state in which user operation for operating the pressure generator 9 is performed, and “OFF” indicates a state in which user operation for stopping the operation of the pressure generator 9 is performed.

Here, before the pressure generator 9 is operated, as illustrated in FIGS. 4A, 5A and 6A, the pressure that is measured by the pressure sensor unit 11 is the pressure P1. Further, when the pressure generator 9 is operated, the pressure that is measured by the pressure sensor unit 11 starts to increase. At this time, a short time interval Tm (FIG. 4A) elapses before the pressure P2 that is generated by the pressure generator 9 propagates to the pressure sensor unit 11. Thereafter, following a time interval Tn (FIG. 4A) until the inside of the treatment tool tube 26 and the inside of the measurement tube TU are completely filled with the pressure P2, increase in the pressure that is measured by the pressure sensor unit 11 is stopped. After the time interval Tn and when the operation of the pressure generator 9 is stopped, the pressure that is measured by the pressure sensor unit 11 starts to decrease after a short delay Tp. When the pressure P2 that is generated by the pressure generator 9 is completely released from the treatment tool tube 26 and the measurement tube TU, the pressure that is measured by the pressure sensor unit 11 reaches the pressure P1 again. In FIG. 4A, a time interval until the pressure P2 decreases to the pressure P1 is described as a time interval Tq.

Further, at Step S3, the processing unit 12 acquires an elapsed time in which the pressure that is measured by the pressure sensor unit 11 meets a predetermined condition, and determines whether or not the residual liquid RL is removed based on the pressure and the elapsed time. More specifically, the processing unit 12 determines that the residual liquid RL is removed when the pressure that is measured by the pressure sensor unit 11 meets the predetermined condition as described above and when the elapsed time as described above exceeds a predetermined threshold Tth1 (FIG. 4A).

In the first embodiment, the predetermined condition as described above is that a variation amount Pd of the pressure that is measured by the pressure sensor unit 11 exceeds a predetermined threshold (P1+Pth1). Here, the variation amount Pd is an amount that is obtained by subtracting the pressure P1 from the pressure P2. In other words, the processing unit 12 acquires, as the elapsed time as described above, an elapsed time Td (FIG. 4A) during which the variation amount Pd exceeds the predetermined threshold (P1+Pth1).

In the example illustrated in FIG. 4A, the variation amount Pd exceeds the predetermined threshold (P1+Pth1) and the elapsed time Td exceeds the threshold Tth1. Therefore, at Step S3, the processing unit 12 determines that the residual liquid RL is removed.

In contrast, in the example illustrated in FIG. 5A, appropriate pressure is not generated by the pressure generator 9, and the variation amount Pd does not exceed the predetermined threshold (P1+Pth1). Therefore, at Step S3, the processing unit 12 determines that the residual liquid RL is not removed.

Furthermore, in the example illustrated in FIG. 6A, the variation amount Pd exceeds the predetermined threshold (P1+Pth1), but an operation time of the pressure generator 9 is short and the elapsed time Td does not exceed the threshold Tth1. Therefore, at Step S3, the processing unit 12 determines that the residual liquid RL is not removed.

After Step S3, the processing unit 12 controls operation of at least one of the image generation unit 14 and the sound emission unit 17, and gives a notice of a result of the process of determining removal of the residual liquid RL from at least one of the display unit 16 and the sound emission unit 17 (Step S4).

FIG. 7 is a diagram illustrating an image F1 that is displayed on the display unit 16 at Step S4. Specifically, FIG. 7 illustrates the image F1 that is displayed on the display unit 16 when it is determined that the residual liquid RL is not removed.

For example, when the processing unit 12 determines, at Step S3, that the residual liquid RL is not removed, controls operation of the image generation unit 14 and causes the display unit 16 to display the image F1 as illustrated in FIG. 7. Meanwhile, it may be possible to cause the display device 8 to display the image F1. Specifically, as illustrated in FIG. 7, the image F1 is an image that includes a message image F11 indicating that “residual liquid removal is not appropriately performed” and an icon I1 for prompting to perform operation for switching to a “diagnosis mode”. The “diagnosis mode” is a mode for displaying information on internal pressure of the lumen LU that is needed for the GERD diagnosis.

Meanwhile, although specific illustration is omitted, when it is determined, at Step S3, that the residual liquid RL is removed, the display unit 16 displays an image that includes a message image indicating that “residual liquid removal is appropriately performed” and the icon I1 as described above.

After Step S4, the processing unit 12 determines whether or not “operation for switching to diagnosis mode (operation on the icon I1 illustrated in FIG. 7)” is performed on the input unit 15 by the user, such as the doctor (Step S5).

When it is determined that the operation for switching to the diagnosis mode is not performed (Step S5: No), the processing unit 12 determines whether or not operation for starting to remove the residual liquid is performed on the input unit 15 by the user, such as the doctor (Step S6).

When it is determined that the operation for starting to remove the residual liquid is not performed (Step S6: No), the processing unit 12 returns to Step S5.

In contrast, when it is determined that the operation for starting to remove the residual liquid is performed (Step S6: Yes), the processing unit 12 returns to Step S2.

In contrast, when it is determined that the operation for switching to the diagnosis mode is performed (Step S5: Yes), the processing unit 12 switches to the “diagnosis mode” (Step S7).

Further, the doctor performs the GERD diagnosis.

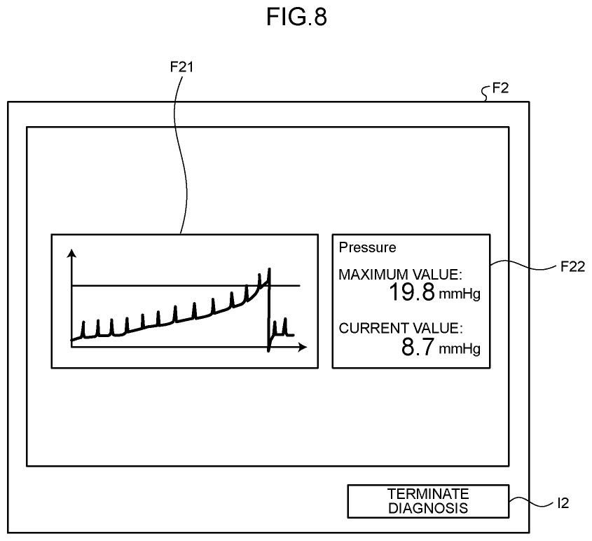

FIG. 8 is a diagram illustrating an image F2 that is displayed on the display unit 16 in the GERD diagnosis.

Specifically, the doctor turns on the gas supply button 221 and causes the gas supply device 4 to perform operation of supply gas into the lumen LU to expand the inside of the lumen LU. In contrast, the processing unit 12 controls operation of the image generation unit 14 and causes the display unit 16 to display an image F2 that includes a waveform image F21 indicating a pressure state inside the lumen LU, a numerical image F22 indicating numerical information on the pressure, and an icon I2 for terminating the GERD diagnosis and prompting to perform operation for switching to the “residual liquid removal monitoring mode”, based on the pressure that is measured by the pressure sensor unit 11. Meanwhile, it may be possible to cause the display device 8 to display the image F2. Furthermore, the doctor performs the GERD diagnosis based on the image F2 and a relaxed state of the lower esophageal sphincter (LES) that is observed from an endoscopic image.

According to the first embodiment as described above, it is possible to achieve effects as described below.

In the pressure measurement apparatus 10 according to the first embodiment, the processing unit 12 acquires the pressure that is measured by the pressure sensor unit 11, acquires the elapsed time Td in which the pressure meets a predetermined condition, and determines whether or not the residual liquid RL in the treatment tool tube 26 is removed based on the pressure and the elapsed time Td. The predetermined condition is that the variation amount Pd of the pressure that is measured by the pressure sensor unit 11 exceeds the predetermined threshold (P1+Pth1). More specifically, the processing unit 12 determines that the residual liquid RL is removed when the variation amount Pd of the pressure that is measured by the pressure sensor unit 11 exceeds the predetermined threshold (P1+Pth1) and the elapsed time Td exceeds the predetermined threshold Tth1.

Therefore, according to the pressure measurement apparatus 10 of the first embodiment, it is possible to appropriately determine whether or not the residual liquid RL is present in the treatment tool tube 26.

Furthermore, in the pressure measurement apparatus 10 according to the first embodiment, the processing unit 12 controls operation of at least one of the image generation unit 14 and the sound emission unit 17, and gives a notice of a result of the process of determining removal of the residual liquid RL from at least one of the display unit 16 and the sound emission unit 17.

Therefore, a user, such as a doctor, is able to clearly determine whether or not the residual liquid RL is present in the treatment tool tube 26. In other words, when the user, such as the doctor, recognizes that the residual liquid RL is present in the treatment tool tube 26 based on the notice that is given by at least one of the display unit 16 and the sound emission unit 17, the user performs the operation of removing the residual liquid RL again. Therefore, it is possible to perform the GERD diagnosis in a state in which the residual liquid RL in the treatment tool tube 26 is infallibly removed.

### Second Embodiment

A second embodiment will be described below.

In the following explanation, the same components as those of the first embodiment as described above are denoted by the same reference symbols, and detailed explanation thereof will be omitted or simplified.

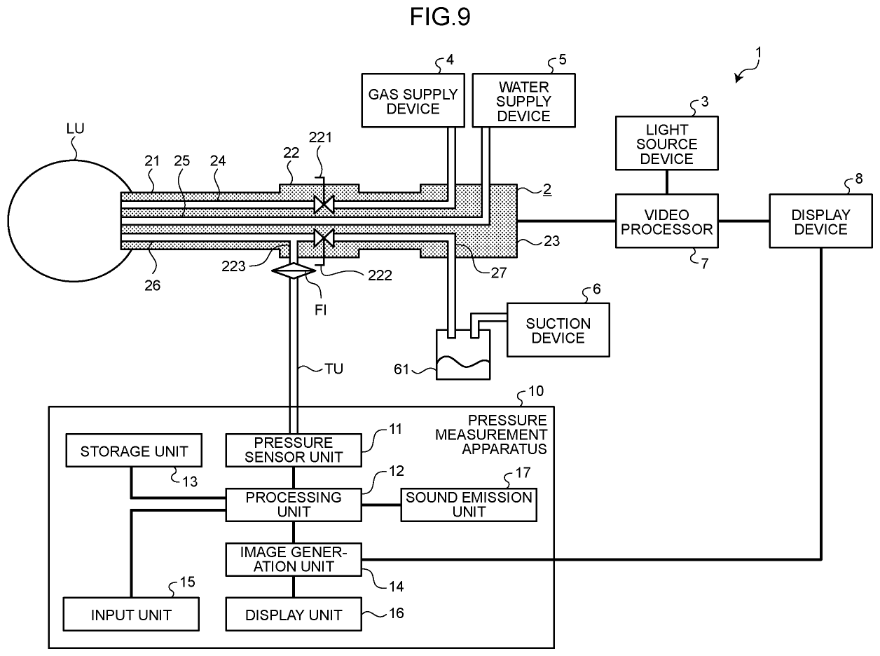

FIG. 9 is a diagram illustrating the endoscope system 1 according to the second embodiment.

In the endoscope system 1 according to the second embodiment, as illustrated in FIG. 9, the suction device 6 is provided with the functions of the pressure generator 9 and the pressure generator 9 is omitted as compared to the endoscope system 1 according to the first embodiment as described above.

Specifically, in the method of measuring pressure inside a body according to the second embodiment, a user, such as a doctor, performs operation on the input unit 15 for starting to remove residual liquid after the insertion portion 21 is inserted from a mouth of a patient to a lower esophageal sphincter (LES). Further, the user, such as the doctor, turns on the suction button 222 to cause the suction device 6 to perform suction operation, and after a lapse of a certain time, turns off the suction button 222 to stop the suction operation.

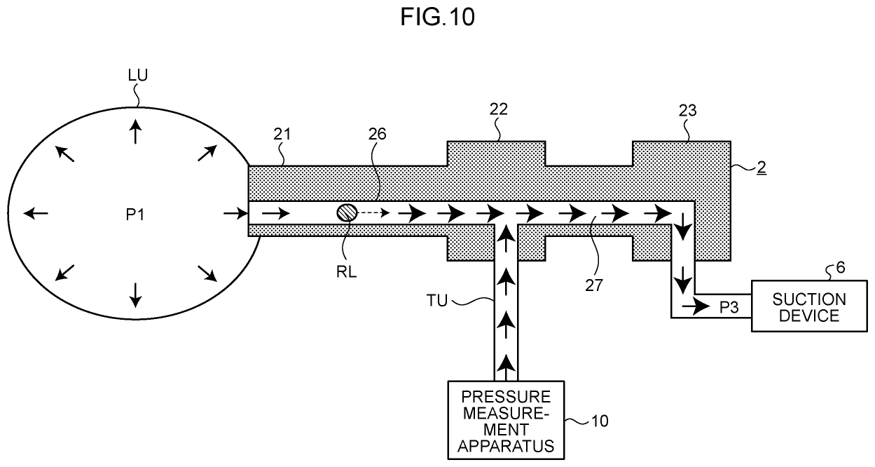

FIG. 10 is a diagram illustrating a pressure propagation state when the suction device 6 performs the suction operation. Specifically, solid arrows indicate propagation directions of pressure. Further, sizes of arrow feathers indicate pressure intensities. Furthermore, a dashed arrow indicates a movement direction of the residual liquid RL.

Here, immediately after the insertion portion 21 is introduced into the lumen LU, the weak pressure P1 (normally, about 0 to 5 mmHg) is generated in the lumen LU. Further, strong pressure P3 (negative pressure) is applied in the suction tube 27 due to the suction device 6. Furthermore, when the suction tube 27 and the treatment tool tube 26 communicate with each other by the suction button 222, the pressure P3 propagates through the inside of the treatment tool tube 26 and the inside the measurement tube TU, and is transmitted to the pressure measurement apparatus 10 and the residual liquid RL in the treatment tool tube 26. Accordingly, kinetic energy is generated on the residual liquid RL, so that the residual liquid RL moves to a lower-pressure side (the pressure P3 side).

The method of measuring pressure inside a body according to the second embodiment is different from the method of measuring pressure inside a body according to the first embodiment as described above in terms of the process of determining removal of the residual liquid RL (Step S3).

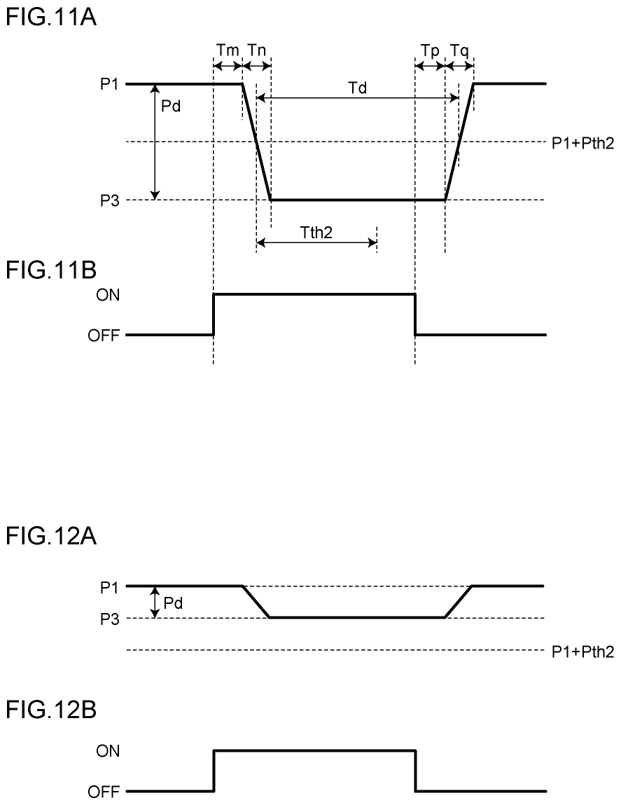

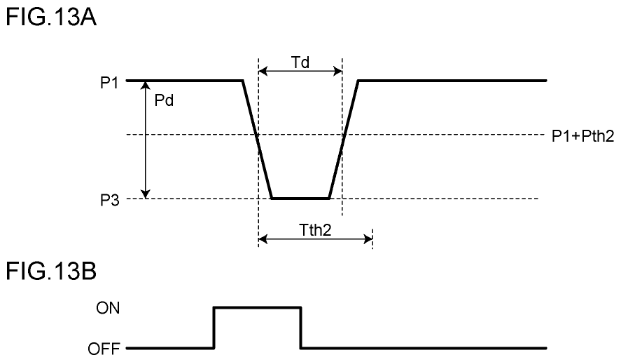

FIG. 11A to FIG. 13B are diagrams for explaining the process of determining removal of the residual liquid RL (Step S3). Specifically, FIG. 11A, FIG. 12A, and FIG. 13A are diagrams illustrating behaviors of the pressure that is measured by the pressure sensor unit 11 when the suction device 6 performs the suction operation. FIG. 11B, FIG. 12B, and FIG. 13B are diagrams illustrating states of the suction operation performed by the suction device 6, where “ON” indicates a state in which the suction operation is performed (a state in which the suction button 222 is turned on), and “OFF” indicates a state in which the suction operation is stopped (a state in which the suction button 222 is turned off).

Here, before the suction operation is performed (before the treatment tool tube 26 and the suction tube 27 communicate with each other by turning on the suction button 222), as illustrated in FIGS. 11A, 12A and 13A, the pressure that is measured by the pressure sensor unit 11 is the pressure P1. Further, when the suction operation is performed, the pressure that is measured by the pressure sensor unit 11 starts to decrease. At this time, the short time interval Tm (FIG. 11A) elapses before the pressure P3 that is generated by the suction device 6 propagates to the pressure sensor unit 11. Thereafter, following the time interval Tn (FIG. 11A) until the inside of the treatment tool tube 26 and the inside of the measurement tube TU are completely filled with the pressure P3, decrease in the pressure that is measured by the pressure sensor unit 11 is stopped. After the time interval Tn and when the suction button 222 is turned off and the suction operation is stopped, the pressure that is measured by the pressure sensor unit 11 starts to increase after the short delay Tp. When the pressure P3 that is generated by the suction device 6 is completely released from the treatment tool tube 26 and the measurement tube TU, the pressure that is measured by the pressure sensor unit 11 reaches the pressure P1 again. In FIG. 11A, a time interval until the pressure P3 increases to the pressure P1 is described as the time interval Tq.

Further, at Step S3, the processing unit 12 acquires an elapsed time in which the pressure that is measured by the pressure sensor unit 11 meets a predetermined condition, and determines whether or not the residual liquid RL is removed based on the pressure and the elapsed time. More specifically, the processing unit 12 determines that the residual liquid RL is removed when the pressure that is measured by the pressure sensor unit 11 meets the predetermined condition as described above and when the elapsed time as described above exceeds a threshold Tth2 (FIG. 11A).

In the second embodiment, the predetermined condition as described above is that the variation amount Pd of the pressure that is measured by the pressure sensor unit 11 falls below a predetermined threshold (P1+Pth2). Here, the variation amount Pd is an amount that is obtained by subtracting the pressure P1 from the pressure P3. In other words, the processing unit 12 acquires, as the elapsed time as described above, the elapsed time Td (FIG. 11A) in which the variation amount Pd falls below the predetermined threshold (P1+Pth2).

In the example illustrated in FIG. 11A, the variation amount Pd falls below the predetermined threshold (P1+Pth2) and the elapsed time Td exceeds the threshold Tth2. Therefore, at Step S3, the processing unit 12 determines that the residual liquid RL is removed.

In contrast, in the example illustrated in FIG. 12A, appropriate pressure is not generated by the suction device 6, and the variation amount Pd does not fall below the predetermined threshold (P1+Pth2). Therefore, at Step S3, the processing unit 12 determines that the residual liquid RL is not removed.

Further, in the example illustrated in FIG. 13A, the variation amount Pd falls below the predetermined threshold (P1+Pth2), but a suction operation time of the suction device 6 is short and the elapsed time Td does not exceed the threshold Tth2. Therefore, at Step S3, the processing unit 12 determines that the residual liquid RL is not removed.

As in the second embodiment as described above, even when the suction device 6 is provided with the functions of the pressure generator 9, it is possible to achieve the same effects as those of the first embodiment as described above.

### Third Embodiment

A third embodiment will be described below.

In the following explanation, the same components as those of the first embodiment as described above are denoted by the same reference symbols, and detailed explanation thereof will be omitted or simplified.

The third embodiment is different from the first embodiment as described above in terms of the process of determining removal of the residual liquid RL (Step S3).

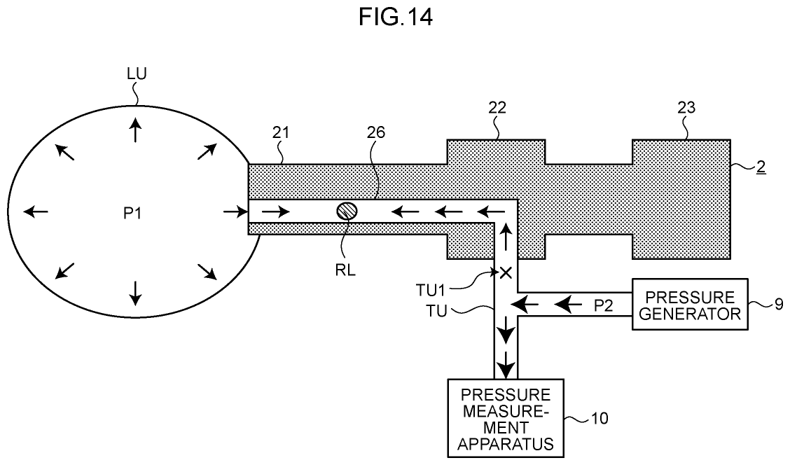

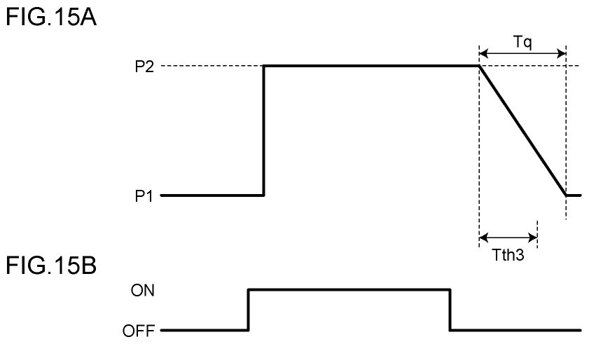

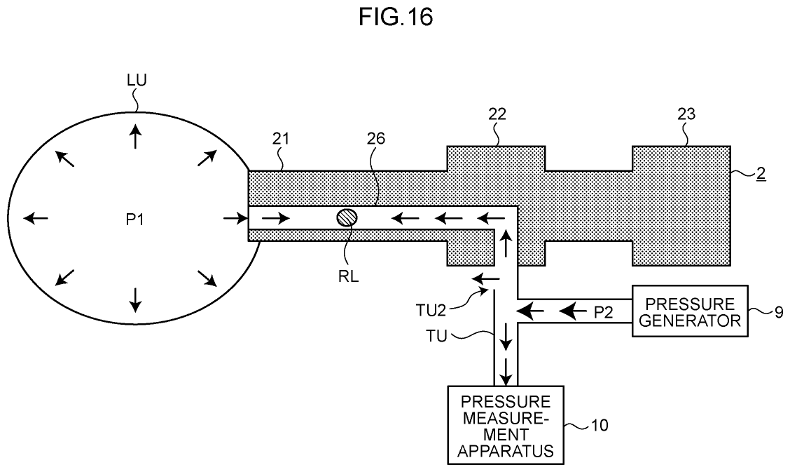

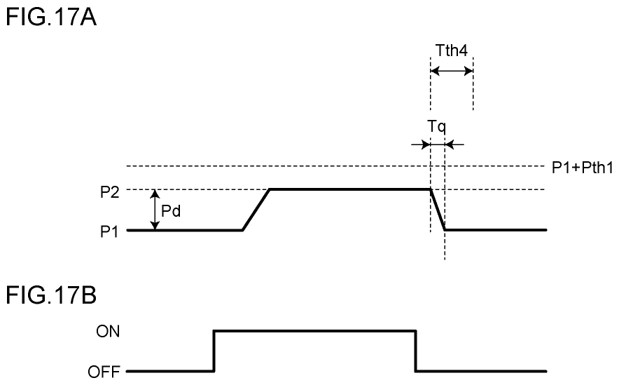

FIG. 14 to FIG. 17B are diagrams for explaining the process of determining removal of the residual liquid RL (Step S3). FIG. 14 is a diagram corresponding to FIG. 3, and illustrates a state in which the measurement tube TU is buckled. In FIG. 14, a portion indicated by “×” represents a buckled portion TU1. FIGS. 15A and 15B are diagrams corresponding to FIG. 14. Specifically, FIG. 15A is a diagram illustrating a behavior of the pressure that is measured by the pressure sensor unit 11 when the pressure generator 9 is operated in a state in which the measurement tube TU is buckled. FIG. 15B is a diagram illustrating an operation state of the pressure generator 9, where “ON” indicates a state in which user operation for operating the pressure generator 9 is performed, and “OFF” indicates a state in which user operation for stopping the operation of the pressure generator 9 is performed. FIG. 16 is a diagram corresponding to FIG. 3, and illustrates a state in which the measurement tube TU includes a leak portion TU2. FIGS. 17A and 17B are diagrams corresponding to FIG. 16. Specifically, FIG. 17A is a diagram illustrating a behavior of the pressure that is measured by the pressure sensor unit 11 when the pressure generator 9 is operated in a state in which the measurement tube TU includes the leak portion TU2. FIG. 17B is a diagram illustrating an operation state of the pressure generator 9, where “ON” indicates a state in which user operation for operating the pressure generator 9 is performed, and “OFF” indicates a state in which user operation for stopping the operation of the pressure generator 9 is performed.

In the third embodiment, the processing unit 12 determines that the residual liquid RL is removed when all of a first condition to a third condition are met. The first condition is that the variation amount Pd of the pressure that is measured by the pressure sensor unit 11 exceeds the threshold (P1+Pth1). The second condition is that the elapsed time Td exceeds the threshold Tth1. The third condition is that the time interval Tq falls within a predetermined range. The time interval Tq corresponds to a first arrival time interval.

In the examples illustrated in FIG. 14 and FIGS. 15A and 15B, the state in which the measurement tube TU is buckled is illustrated. When the measurement tube TU is buckled as described above, in some cases, the first condition and the second condition as described may be met. In other words, if it is determined that the residual liquid RL is removed based on only the first condition and the second condition, in some cases, it may be determined that the residual liquid RL is removed even when the pressure P2 that is generated by the pressure generator 9 does not propagate through the inside of the treatment tool tube 26 and the residual liquid RL is not moved (removed) due to the buckling of the measurement tube TU.

Further, when the measurement tube TU is buckled, the time interval Tq is relatively long. Therefore, in the third embodiment, the processing unit 12 determines that the residual liquid RL is removed when not only the first condition and the second condition, but also the third condition are met. Specifically, in the example illustrated in FIG. 15A, the time interval Tq exceeds an upper limit value Tth3 of a predetermined range as the third condition, and therefore, the processing unit 12 determines, at Step S3, that the residual liquid RL is not removed.

In the examples illustrated in FIG. 16 and FIGS. 17A and 17B, the state in which the measurement tube TU includes the leak portion TU2 is illustrated. When the measurement tube TU includes the leak portion TU2 as described above, in some cases, the first condition and the second condition as described may be met. In other words, if it is determined that the residual liquid RL is removed based on only the first condition and the second condition, in some cases, it may be determined that the residual liquid RL is removed even when the pressure P2 that is generated by the pressure generator 9 does not propagate through the inside of the treatment tool tube 26 and the residual liquid RL is not moved (removed) due to the leak portion TU2.

Further, when the measurement tube TU includes the leak portion TU2, the time interval Tq is relatively short. Therefore, in the third embodiment, the processing unit 12 determines that the residual liquid RL is removed when not only the first condition and the second condition, but also the third condition are met. Specifically, in the example illustrated in FIG. 17A, the time interval Tq is shorter than a lower limit value Tth4 of the predetermined range as the third condition, and therefore, the processing unit 12 determines, at Step S3, that the residual liquid RL is not removed.

According to the third embodiment as described above, it is possible to achieve effects as described below, in addition to the same effects as those of the first embodiment as described above.

In the pressure measurement apparatus 10 according to the third embodiment, the processing unit 12 determines that the residual liquid RL is removed when all of the first condition to the third condition as described above are met.

Therefore, even when the measurement tube TU is buckled or the measurement tube TU includes the leak portion TU2, it is possible to appropriately determine whether or not the residual liquid RL is removed.

### Fourth Embodiment

A fourth embodiment will be described below.

In the following explanation, the same components as those of the first embodiment as described above are denoted by the same reference symbols, and detailed explanation thereof will be omitted or simplified.

The endoscope system 1 according to the fourth embodiment is different from the endoscope system 1 according to the first embodiment as described above in that, at Step S4, at least one of the display unit 16 (the display device 8) and the sound emission unit 17 gives a notice of a result of the process of determining removal of the residual liquid RL by a different notification method.

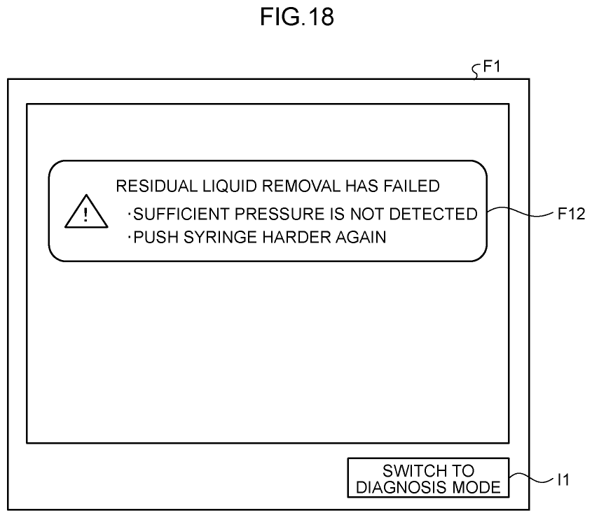

FIG. 18 is a diagram illustrating the image F1 that is displayed by the display unit 16 at Step S4 according to the fourth embodiment. Specifically, FIG. 18 corresponds to FIG. 7, and illustrates the image F1 that is displayed by the display unit 16 when it is determined that the residual liquid RL is not removed. More specifically, FIG. 18 illustrates the image F1 that is displayed by the display unit 16 when the pressure generator 9 is configured with a syringe that sends out a predetermined amount of gas by manual operation and when the variation amount Pd does not exceed the threshold (P1+Pth1) (FIG. 5).

The processing unit 12 according to the fourth embodiment, when determining that, at Step S3, the residual liquid RL is not removed, controls operation of the image generation unit 14 and gives a notice of information on a determination reason and a coping method from at least one of the display unit 16 and the sound emission unit 17. In the example illustrated in FIG. 18, a case is illustrated in which the image F1 that includes the information on the determination reason and the coping method is displayed by the display unit 16. Meanwhile, it may be possible to display the image F1 by the display device 8.

For example, when the variation amount Pd does not exceed the threshold (P1+Pth1) (FIG. 5) and when determining that the residual liquid RL is not removed, the processing unit 12 controls operation of the image generation unit 14 and causes the display unit 16 to display the image F1 as illustrated in FIG. 18. Specifically, as illustrated in FIG. 18, the image F1 is an image that includes the message image F12 indicating a determination result such as “residual liquid removal has failed”, a determination reason such as “sufficient pressure is not detected”, and a coping method such as “push syringe harder again”, and the icon I1 for prompting to perform operation of switching to the “diagnosis mode”.

Meanwhile, although specific illustration is omitted, when the elapsed time Td does not exceed the threshold Tth1 (FIG. 6A) and when determining that the residual liquid RL is not removed, the processing unit 12 causes the display unit 16 to display the image F1 that includes a message image indicating that, for example “residual liquid removal has failed” and a coping method such as “push syringe harder again”, and the icon I1 as described above.

According to the fourth embodiment as described above, it is possible to achieve effects as described below, in addition to the same effects as those of the first embodiment as described above.

In the pressure measurement apparatus 10 according to the fourth embodiment, when a determination result indicates that the residual liquid RL is not removed in the process of determining removal of the residual liquid RL (Step S3), the processing unit 12 gives a notice of information on the determination reason or the coping method from at least one of the display unit 16 and the sound emission unit 17.

Therefore, the user, such as the doctor, is able to appropriately recognize a cause for a failure to remove the residual liquid RL, so that it is possible to accurately perform the operation of removing the residual liquid RL again.

### Fifth Embodiment

A fifth embodiment will be described below.

In the following explanation, the same components as those of the first embodiment as described above are denoted by the same reference symbols, and detailed explanation thereof will be omitted or simplified.

The fifth embodiment is different from the first embodiment as described above in terms of the method of measuring pressure inside a body.

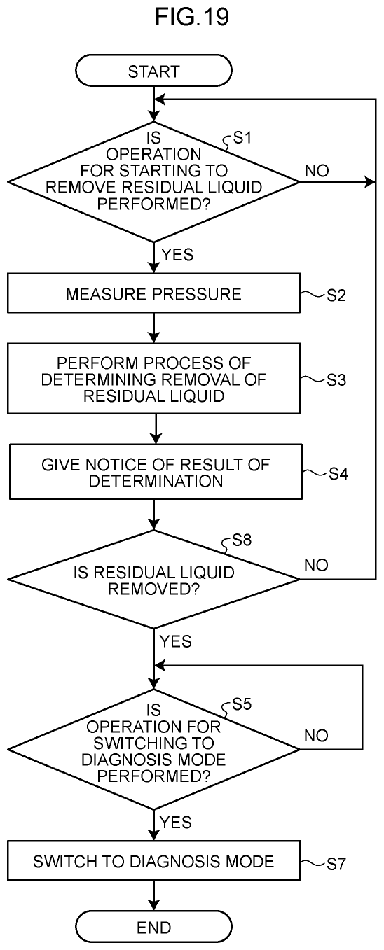

FIG. 19 is a flowchart illustrating the method of measuring pressure inside a body according to the fifth embodiment. Specifically, FIG. 19 is a diagram corresponding to FIG. 2, and is a flowchart illustrating a process that is performed by the processing unit 12.

In the method of measuring pressure inside a body according to the fifth embodiment, as illustrated in FIG. 19, Step S6 is omitted and Step S8 is added in the method of measuring pressure inside a body according to the first embodiment as described above. In the following, Step S8 will be mainly described.

Step S8 is performed after Step S4.

Specifically, at Step S8, the processing unit 12 determines whether or not it is determined that the residual liquid RL is removed in the process of determining removal of the residual liquid RL (Step S3).

When it is determined that the residual liquid RL is not removed (Step S8: No), the processing unit 12 returns to Step S1.

In contrast, when it is determined that the residual liquid RL is removed (Step S8: Yes), the processing unit 12 goes to Step S5.

Further, at Step S5, the processing unit 12 continuously monitors whether or not the user, such as the doctor, performs, on the input unit 15, the “operation for switching to the diagnosis mode” (operation on the icon I1 illustrated in FIG. 7)”.

When it is determined that the operation for switching to the diagnosis mode is performed (Step S5: Yes), the processing unit 12 goes to Step S7.

As described above, in the fifth embodiment, when determining that the residual liquid RL is not removed, the processing unit 12 restricts switching to the diagnosis mode.

According to the fifth embodiment as described above, it is possible to achieve effects as described below, in addition to the same effects as those of the first embodiment as described above.

In the pressure measurement apparatus 10 according to the fifth embodiment, when determining that the residual liquid RL is not removed, the processing unit 12 restricts switching to the diagnosis mode.

Therefore, it is possible to prevent a situation in which the GERD diagnosis is erroneously started even when the residual liquid RL is not removed.

### Sixth Embodiment

A sixth embodiment will be described below.

In the following explanation, the same components as those of the first embodiment as described above are denoted by the same reference symbols, and detailed explanation thereof will be omitted or simplified.

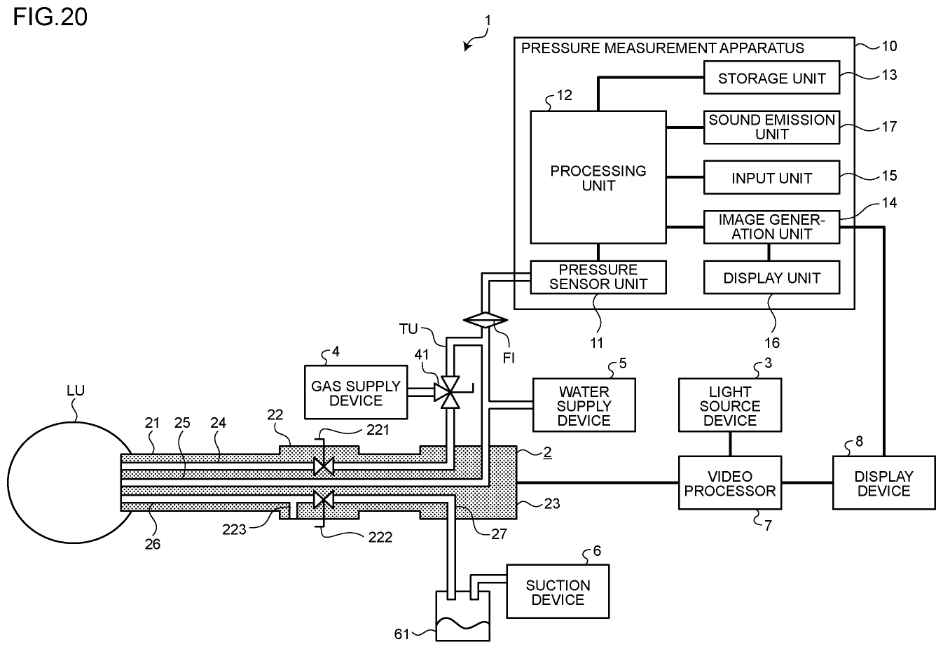

FIG. 20 is a diagram illustrating the endoscope system 1 according to the sixth embodiment.

In the endoscope system 1 according to the sixth embodiment, as illustrated in FIG. 20, the gas supply device 4 is provided with the functions of the pressure generator 9 and the pressure generator 9 is omitted as compared to the endoscope system 1 according to the first embodiment as described above. Further, in the endoscope system 1 according to the sixth embodiment, the measurement tube TU is connected to the water supply tube 25 instead of the treatment tool tube 26. Further, in the endoscope system 1 according to the sixth embodiment, the water supply device 5 is connected to the water supply tube 25 via the measurement tube TU. Furthermore, in the endoscope system 1 according to the sixth embodiment, the gas supply device 4 is connected to the gas supply tube 24 and the measurement tube TU by a three-way valve 41. Specifically, by switching the three-way valve 41, the gas supply device 4 communicates with one of the gas supply tube 24 and the measurement tube TU. Moreover, the water supply tube 25 and the measurement tube TU correspond to the pressure measurement tube.

Specifically, in the method of measuring pressure inside a body according to the sixth embodiment, a user, such as a doctor, performs operation on the input unit 15 for starting to remove residual liquid after the insertion portion 21 is inserted from a mouth of a patient to a lower esophageal sphincter (LES). Further, the user, such as the doctor, allows communication between the gas supply device 4 and the measurement tube TU by the three-way valve 41, turns on the gas supply button 221 to cause the gas supply device 4 to perform gas supply operation through the measurement tube TU and the water supply tube 25, and, after a lapse of a certain time, turns off the gas supply button 221 to stop the gas supply operation.

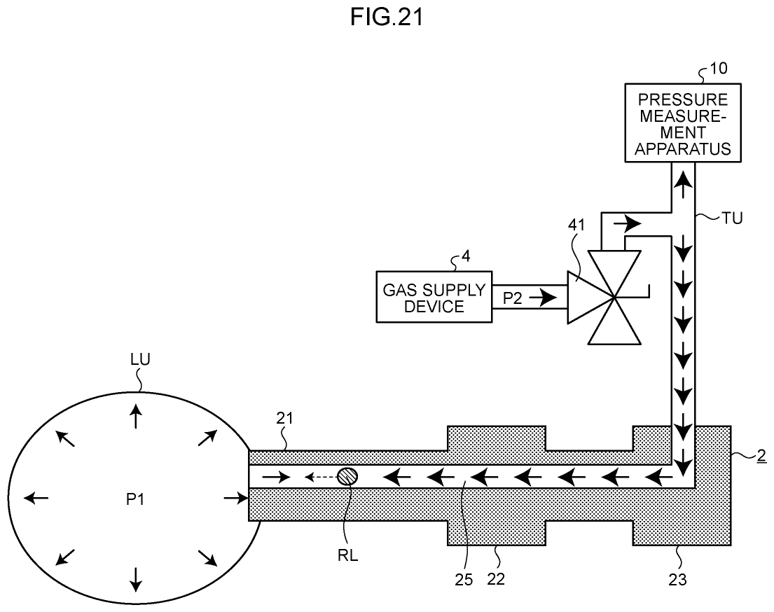

FIG. 21 is a diagram illustrating a pressure propagation state when the gas supply device 4 performs the gas supply operation. Specifically, solid arrows indicate propagation directions of pressure. Further, sizes of arrow feathers indicate pressure intensities. Furthermore, a dashed arrow indicates a movement direction of the residual liquid RL.

Here, immediately after the insertion portion 21 is introduced into the lumen LU, the weak pressure P1 (normally, about 0 to 5 mmHg) is generated in the lumen LU. Further, when the three-way valve 41 is switched to allow communication between the gas supply device 4 and the measurement tube TU and the gas supply button 221 is turned on, the pressure P2 that is generated by the gas supply device 4 propagates through the inside of the measurement tube TU and the inside of the water supply tube 25, and is transmitted to the pressure measurement apparatus 10 and the residual liquid RL inside the water supply tube 25. Accordingly, kinetic energy is generated on the residual liquid RL, so that the residual liquid RL moves to a lower-pressure side (the pressure P1 side).

Meanwhile, in the method of measuring pressure inside a body according to the sixth embodiment, the process that is performed by the processing unit 12 is the same as the process (Step S1 to Step S7) of the first embodiment as described above.

As in the sixth embodiment as described above, even when the gas supply device 4 is provided with the functions of the pressure generator 9, it is possible to achieve the same effects as those of the first embodiment as described above.

### Seventh Embodiment

A seventh embodiment will be described below.

In the following explanation, the same components as those of the sixth embodiment as described above are denoted by the same reference symbols, and detailed explanation thereof will be omitted or simplified.

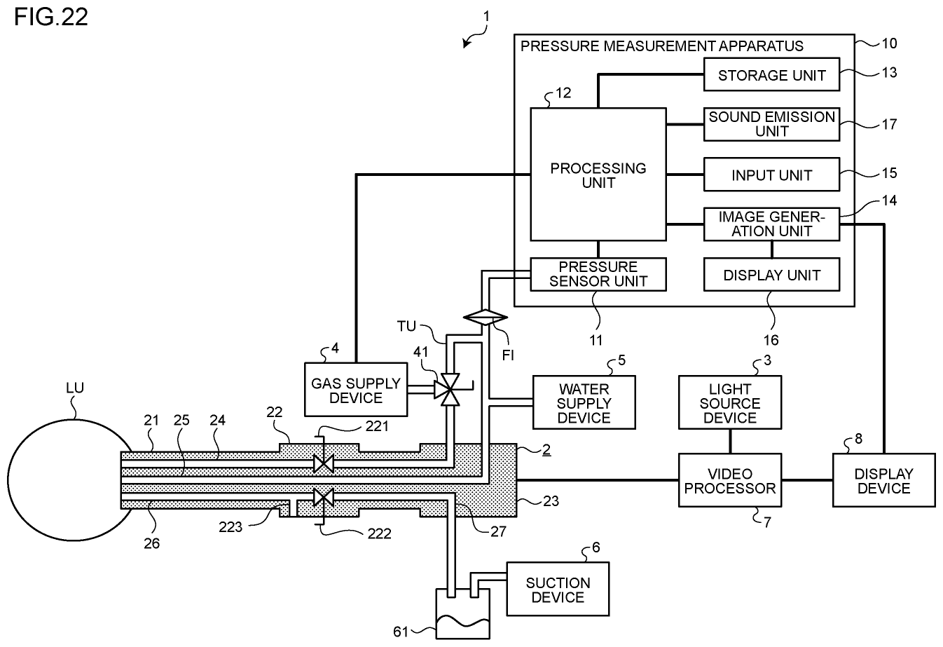

FIG. 22 is a diagram illustrating the endoscope system 1 according to the seventh embodiment.

The endoscope system 1 according to the seventh embodiment is different from the endoscope system 1 according to the sixth embodiment as described above in that, as illustrated in FIG. 22, the gas supply device 4 and the pressure measurement apparatus 10 are electrically connected to each other. Further, the pressure measurement apparatus 10 is able to recognize a point of time of ON and OFF of gas supply operation performed by the gas supply device 4. Furthermore, the endoscope system 1 according to the seventh embodiment is different from the endoscope system 1 according to the sixth embodiment as described above in terms of the process of determining removal of the residual liquid RL (Step S3).

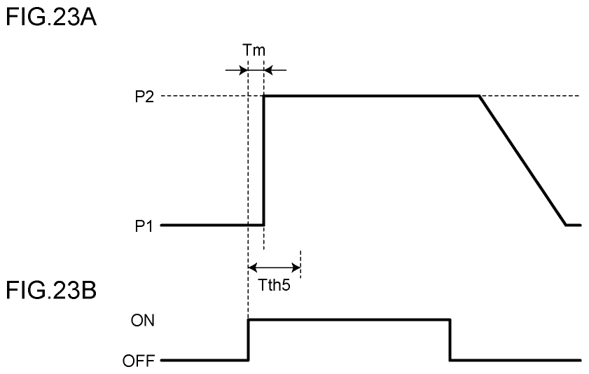

FIGS. 23A and 23B are diagrams for explaining the process of determining removal of the residual liquid RL (Step S3). FIGS. 23A and 23B are diagrams corresponding to FIG. 4A to FIG. 6B. Specifically, FIG. 23A is a diagram illustrating a behavior of the pressure that is measured by the pressure sensor unit 11 when the gas supply device 4 performs gas supply operation in a state in which the measurement tube TU is buckled. FIG. 23B is a diagram illustrating a state of the gas supply operation that is performed by the gas supply device 4, where “ON” indicates a state in which the gas supply operation is performed, and “OFF” indicates a state in which the gas supply operation is stopped.

In the seventh embodiment, the processing unit 12 determines that the residual liquid RL is removed when all of a first condition, a second condition, and a fourth condition are met. The first condition is that the variation amount Pd of the pressure that is measured by the pressure sensor unit 11 exceeds the threshold (P1+Pth1). The second condition is that the elapsed time Td exceeds the threshold Tth1. The fourth condition is that the time interval Tm is equal to or shorter than a predetermined threshold Tth5. The time interval Tm is a time from when the gas supply device 4 starts the gas supply operation to when the pressure that is measured by the pressure sensor unit 11 reaches the pressure P2. The time interval Tm corresponds to a second arrival time interval.

In the example illustrated in FIGS. 23A and 23B, the state in which the measurement tube TU is buckled is illustrated. When the measurement tube TU is buckled as described above, in some cases, the first condition and the second condition as described may be met. In other words, if it is determined that the residual liquid RL is removed based on only the first condition and the second condition, in some cases, it may be determined that the residual liquid RL is removed even when the pressure P2 that is generated by the pressure generator 9 does not propagate through the inside of the water supply tube 25 and the residual liquid RL is not moved (removed) due to the buckling of the measurement tube TU.

Further, when the measurement tube TU is buckled, the time interval Tm is relatively short. Therefore, in the seventh embodiment, the processing unit 12 determines that the residual liquid RL is removed when not only the first condition and the second condition, but also the fourth condition are met. Specifically, in the example illustrated in FIG. 23A, the time interval Tm is equal to or shorter than the predetermined threshold Tth5, and therefore, the processing unit 12 determines, at Step S3, that the residual liquid RL is not removed.

According to the seventh embodiment as described above, it is possible to achieve effects as described below, in addition to the same effects as those of the sixth embodiment as described above.

In the pressure measurement apparatus 10 according to the seventh embodiment, the processing unit 12 determines that the residual liquid RL is removed when all of the first condition, the second condition, and the fourth condition as described above are met.

Therefore, even when the measurement tube TU is buckled, it is possible to appropriately determine whether or not the residual liquid RL is removed.

### Other Embodiments

While the embodiments for carrying out the present invention have been described above, the present invention is not limited to only the first embodiment to the seventh embodiment as described above.

In the first embodiment to the seventh embodiment as described above, the pressure Pth1, the pressure Pth2, the thresholds Tth1, Tth2, and Tth5, the upper limit value Tth3, and the lower limit value Tth4 may be set to appropriate values based on a cross-sectional area of the pressure measurement tube, the pressure (P2 or P3) generated in the pressure measurement tube, frictional force that occurs between the pressure measurement tube and the residual liquid RL, an expected mass of the residual liquid RL, and the like.

According to a control apparatus, an endoscope system, and a method of measuring pressure inside a body of the disclosure, it is possible to appropriately determine whether or not a foreign object is present in a pressure measurement tube of an endoscope.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

### EXAMPLES

### Example 1

A control apparatus comprising:
at least a single processor configured to
   acquire pressure in a tube of an endoscope when the pressure in the tube is changed to remove a foreign object from inside the tube,
   acquire an elapsed time in which the pressure in the tube meets a predetermined condition, and
   determine whether or not the foreign object in the tube is removed based on the pressure in the tube and the elapsed time.

### Example 2

The control apparatus according to Example 1, wherein the processor is further configured to determine that the foreign object in the tube is removed when the pressure in the tube meets the predetermined condition and the elapsed time exceeds a predetermined threshold.

### Example 3

The control apparatus according to Example 1, wherein the processor is further configured to
acquire a first arrival time interval from when a change of the pressure in the tube is terminated to when the pressure in the tube reaches predetermined pressure, and
determine whether or not the foreign object in the tube is removed based on the pressure in the tube, the elapsed time, and the first arrival time interval.

### Example 4

The control apparatus according to Example 1, wherein the processor is further configured to
acquire a second arrival time interval from when a change of the pressure in the tube is started to when the pressure in the tube reaches predetermined pressure, and
determine whether or not the foreign object in the tube is removed based on the pressure in the tube, the elapsed time, and the second arrival time interval.

### Example 5

The control apparatus according to Example 1, wherein the processor is further configured to cause a notification unit to give a notice of determination result information on a result of determination on whether or not the foreign object in the tube is removed.

### Example 6

The control apparatus according to Example 5, wherein the determination result information includes information on a determination reason or a coping method when the result of determination indicates that the foreign object in the tube is not removed.

### Example 7

The control apparatus according to Example 1, wherein the processor is further configured to restrict switching to a mode for performing diagnosis when it is determined that the foreign object in the tube is not removed.

### Example 8

An endoscope system comprising:
an endoscope that includes a first tube for measuring pressure inside a body;
a second tube that is connected to the first tube;
a pressure sensor that is connected to the second tube; and
a control apparatus that includes at least a single processor, wherein
the processor is configured to
   acquire pressure in the first tube when the pressure in the first tube is changed to remove a foreign object from inside the first tube,
   acquire an elapsed time in which the pressure in the first tube meets a predetermined condition, and
   determine whether or not the foreign object in the first tube is removed based on the pressure in the first tube and the elapsed time.

### Example 9

The endoscope system according to Example 8, wherein the processor is configured to determine that the foreign object in the first tube is removed when the pressure in the first tube meets the predetermined condition and the elapsed time exceeds a predetermined first threshold.

### Example 10

The endoscope system according to Example 8, further comprising:
a display configured to display a predetermined image, wherein the processor is further configured to cause the display to display determination result information on a result of determination on whether or not the foreign object in the first tube is removed.

### Example 11

A method of measuring pressure inside a body implemented by a processor of a control apparatus, the method comprising:
acquiring pressure in a tube of an endoscope when the pressure in the tube is changed to remove a foreign object from inside the tube;
acquiring an elapsed time in which the pressure in the tube meets a predetermined condition; and
determining whether or not the foreign object in the tube is removed based on the pressure in the tube and the elapsed time.

### Example 12

The method according to Example 11, wherein the determining includes determining that the foreign object in the tube is removed when the pressure in the tube meets the predetermined condition and the elapsed time exceeds a predetermined threshold.

### Example 13

The method according to Example 11, further comprising acquiring a first arrival time interval from when a change of the pressure in the tube is terminated to when the pressure in the tube reaches predetermined pressure, wherein
the determining includes determining whether or not the foreign object in the tube is removed based on the pressure in the tube, the elapsed time, and the first arrival time interval.

### Example 14

The method according to Example 11, further comprising acquiring a second arrival time interval from when a change of the pressure in the tube is started to when the pressure in the tube reaches predetermined pressure, wherein
the determining includes determining whether or not the foreign object in the tube is removed based on the pressure in the tube, the elapsed time, and the second arrival time interval.

### Example 15

The method according to Example 11, further comprising causing a notification unit to give a notice of determination result information on a result of determination on whether or not the foreign object in the tube is removed.

### Example 16

The method according to Example 15, wherein the determination result information includes information on a determination reason or a coping method when the result of determination indicates that the foreign object in the tube is not removed.

### Example 17

The method according to Example 11, wherein further comprising restricting switching to a mode for performing diagnosis when it is determined that the foreign object in the tube is not removed.

### Example 18

A control apparatus comprising:
a processor comprising hardware, the processor being configured to
   acquire second pressure in a tube of an endoscope when first pressure in the tube is changed to remove a foreign object from inside the tube,
   acquire an elapsed time in which the second pressure in the tube meets a predetermined condition, and
   determine whether or not the foreign object in the tube is removed based on the second pressure in the tube and the elapsed time.

### Example 19

The control apparatus according to Example 18, wherein the processor is further configured to determine that the foreign object in the tube is removed when the second pressure in the tube meets the predetermined condition and the elapsed time exceeds a predetermined threshold.

### Example 20

The control apparatus according to Example 18, wherein the processor is further configured to
acquire a first arrival time interval during which the second pressure in the tube reaches fourth pressure from third pressure, and
determine whether or not the foreign object in the tube is removed based on the second pressure in the tube, the elapsed time, and the first arrival time interval.

### Example 21

The control apparatus according to Example 18, wherein the processor is further configured to
acquire a second arrival time interval from when an operation for change of the second pressure in the tube is started to when the second pressure in the tube reaches fifth pressure, and
determine whether or not the foreign object in the tube is removed based on the second pressure in the tube, the elapsed time, and the second arrival time interval.

### Example 22

The control apparatus according to Example 18, wherein the processor is further configured to cause a notification unit to give a notice of determination result information on a result of determination on whether or not the foreign object in the tube is removed.

### Example 23

The control apparatus according to Example 22, wherein the determination result information includes information on a determination reason or a coping method when the result of determination indicates that the foreign object in the tube is not removed.

### Example 24

The control apparatus according to Example 18, wherein the processor is further configured to restrict switching to a mode for performing diagnosis when it is determined that the foreign object in the tube is not removed.

### Example 25

The control apparatus according to Example 18, wherein the processor is further configured to
acquire the first pressure and the second pressure in the tube, the first pressure and the second pressure being pressures before and after pressure in the tube is changed to remove the foreign object from inside the tube, respectively, and
determine that the foreign object in the tube has been removed when an absolute value of a difference between the first pressure and the second pressure exceeds a first threshold, and the elapsed time exceeds a second threshold.

### Example 26

The control apparatus according to Example 25, wherein the processor is further configured to
acquire a first arrival time interval during which the second pressure in the tube reaches fourth pressure from third pressure, and
determine that the foreign object in the tube has been removed when the absolute value exceeds the first threshold, the elapsed time exceeds the second threshold, and the first arrival time interval meets a predetermined condition.

### Example 27

The control apparatus according to Example 25, wherein the processor is further configured to
acquire a second arrival time interval from when an operation for change of the first pressure in the tube to when the second pressure in the tube reaches fifth pressure, and
determine that the foreign object in the tube has been removed when the absolute value exceeds the first threshold, the elapsed time exceeds the second threshold, and the second arrival time interval meets a predetermined condition.

### Example 28

An endoscope system comprising:
an endoscope that includes a first tube for measuring pressure inside a body;
a second tube that is connected to the first tube;
a pressure sensor that is connected to the second tube; and
a control apparatus that includes a processor comprising hardware, the processor being configured to
   acquire second pressure in the first tube when first pressure in the first tube is changed to remove a foreign object from inside the first tube,
   acquire an elapsed time in which the second pressure in the first tube meets a predetermined condition, and
   determine whether or not the foreign object in the first tube is removed based on the second pressure in the first tube and the elapsed time.

### Example 29

The endoscope system according to Example 28, wherein the processor is configured to determine that the foreign object in the first tube is removed when the second pressure in the first tube meets the predetermined condition and the elapsed time exceeds a predetermined threshold.

### Example 30

The endoscope system according to Example 28, further comprising:
a display configured to display a predetermined image, wherein the processor is further configured to cause the display to display determination result information on a result of determination on whether or not the foreign object in the first tube is removed.

### Example 31

A method of measuring pressure inside a body, the method comprising:
acquiring second pressure in a tube of an endoscope when first pressure in the tube is changed to remove a foreign object from inside the tube;
acquiring an elapsed time in which the second pressure in the tube meets a predetermined condition; and
determining whether or not the foreign object in the tube is removed based on the second pressure in the tube and the elapsed time.

### Example 32

The method according to Example 31, wherein the determining includes determining that the foreign object in the tube is removed when the second pressure in the tube meets the predetermined condition and the elapsed time exceeds a predetermined threshold.

### Example 33

The method according to Example 31, further comprising acquiring a first arrival time interval during which the second pressure in the tube reaches fourth pressure from third pressure, wherein
the determining includes determining whether or not the foreign object in the tube is removed based on the second pressure in the tube, the elapsed time, and the first arrival time interval.

### Example 34

The method according to Example 31, further comprising acquiring a second arrival time interval from when an operation for change of the first pressure in the tube is started to when the second pressure in the tube reaches fifth pressure, wherein
the determining includes determining whether or not the foreign object in the tube is removed based on the second pressure in the tube, the elapsed time, and the second arrival time interval.

### Example 35

The method according to Example 31, further comprising causing a notification unit to give a notice of determination result information on a result of determination on whether or not the foreign object in the tube is removed.

### Example 36

The method according to Example 35, wherein the determination result information includes information on a determination reason or a coping method when the result of determination indicates that the foreign object in the tube is not removed.

### Example 37

The method according to Example 31, wherein further comprising restricting switching to a mode for performing diagnosis when it is determined that the foreign object in the tube is not removed.

What is claimed is:

1. A control apparatus comprising:
a processor comprising hardware, the processor being configured to
acquire second pressure in a tube of an endoscope when first pressure in the tube is changed to remove a foreign object from inside the tube,
acquire an elapsed time in which the second pressure in the tube meets a predetermined condition, and
determine whether or not the foreign object in the tube is removed based on the second pressure in the tube and the elapsed time.

2. The control apparatus according to claim 1, wherein the processor is further configured to determine that the foreign object in the tube is removed when the second pressure in the tube meets the predetermined condition and the elapsed time exceeds a predetermined threshold.

3. The control apparatus according to claim 1, wherein the processor is further configured to
acquire a first arrival time interval during which the second pressure in the tube reaches fourth pressure from third pressure, and
determine whether or not the foreign object in the tube is removed based on the second pressure in the tube, the elapsed time, and the first arrival time interval.

4. The control apparatus according to claim 1, wherein the processor is further configured to
acquire a second arrival time interval from when an operation for change of the second pressure in the tube is started to when the second pressure in the tube reaches fifth pressure, and
determine whether or not the foreign object in the tube is removed based on the second pressure in the tube, the elapsed time, and the second arrival time interval.

5. The control apparatus according to claim 1, wherein the processor is further configured to cause a notification unit to give a notice of determination result information on a result of determination on whether or not the foreign object in the tube is removed.

6. The control apparatus according to claim 5, wherein the determination result information includes information on a determination reason or a coping method when the result of determination indicates that the foreign object in the tube is not removed.

7. The control apparatus according to claim 1, wherein the processor is further configured to restrict switching to a mode for performing diagnosis when it is determined that the foreign object in the tube is not removed.

8. The control apparatus according to claim 1, wherein the processor is further configured to
acquire the first pressure and the second pressure in the tube, the first pressure and the second pressure being pressures before and after pressure in the tube is changed to remove the foreign object from inside the tube, respectively, and
determine that the foreign object in the tube has been removed when an absolute value of a difference between the first pressure and the second pressure exceeds a first threshold, and the elapsed time exceeds a second threshold.

9. The control apparatus according to claim 8, wherein the processor is further configured to
acquire a first arrival time interval during which the second pressure in the tube reaches fourth pressure from third pressure, and
determine that the foreign object in the tube has been removed when the absolute value exceeds the first threshold, the elapsed time exceeds the second threshold, and the first arrival time interval meets a predetermined condition.

10. The control apparatus according to claim 8, wherein the processor is further configured to
acquire a second arrival time interval from when an operation for change of the first pressure in the tube to when the second pressure in the tube reaches fifth pressure, and
determine that the foreign object in the tube has been removed when the absolute value exceeds the first threshold, the elapsed time exceeds the second threshold, and the second arrival time interval meets a predetermined condition.

11. An endoscope system comprising:
an endoscope that includes a first tube for measuring pressure inside a body;
a second tube that is connected to the first tube;
a pressure sensor that is connected to the second tube; and
a control apparatus that includes a processor comprising hardware, the processor being configured to
acquire second pressure in the first tube when first pressure in the first tube is changed to remove a foreign object from inside the first tube,
acquire an elapsed time in which the second pressure in the first tube meets a predetermined condition, and
determine whether or not the foreign object in the first tube is removed based on the second pressure in the first tube and the elapsed time.

12. The endoscope system according to claim 11, wherein the processor is configured to determine that the foreign object in the first tube is removed when the second pressure in the first tube meets the predetermined condition and the elapsed time exceeds a predetermined threshold.

13. The endoscope system according to claim 11, further comprising:
a display configured to display a predetermined image, wherein the processor is further configured to cause the display to display determination result information on a result of determination on whether or not the foreign object in the first tube is removed.

14. A method of measuring pressure inside a body, the method comprising:
acquiring second pressure in a tube of an endoscope when first pressure in the tube is changed to remove a foreign object from inside the tube;
acquiring an elapsed time in which the second pressure in the tube meets a predetermined condition; and
determining whether or not the foreign object in the tube is removed based on the second pressure in the tube and the elapsed time.

15. The method according to claim 14, wherein the determining includes determining that the foreign object in the tube is removed when the second pressure in the tube meets the predetermined condition and the elapsed time exceeds a predetermined threshold.

16. The method according to claim 14, further comprising acquiring a first arrival time interval during which the second pressure in the tube reaches fourth pressure from third pressure, wherein
the determining includes determining whether or not the foreign object in the tube is removed based on the second pressure in the tube, the elapsed time, and the first arrival time interval.

17. The method according to claim 14, further comprising acquiring a second arrival time interval from when an operation for change of the first pressure in the tube is started to when the second pressure in the tube reaches fifth pressure, wherein
the determining includes determining whether or not the foreign object in the tube is removed based on the second pressure in the tube, the elapsed time, and the second arrival time interval.

18. The method according to claim 14, further comprising causing a notification unit to give a notice of determination result information on a result of determination on whether or not the foreign object in the tube is removed.

19. The method according to claim 18, wherein the determination result information includes information on a determination reason or a coping method when the result of determination indicates that the foreign object in the tube is not removed.

20. The method according to claim 14, wherein further comprising restricting switching to a mode for performing diagnosis when it is determined that the foreign object in the tube is not removed.
